(19) United States
(12) Patent Application Publication
(10) Pub. No.: US 2025/0059142 A1
(43) Pub. Date: Feb. 20, 2025

(54) Method of preparing covalent organic frameworks

(71) Applicant: National Tsing Hua University, HSINCHU (TW)

(72) Inventors: HO-HSIU CHOU, HSINCHU (TW); Ahmed-Mahmoud Elewa, HSINCHU (TW)

(21) Appl. No.: 18/242,802

(22) Filed: Sep. 6, 2023

(30) Foreign Application Priority Data
Aug. 18, 2023 (TW) 112131186

(51) Int. Cl. C07D 251/24; C07C 251/24; C07D 471/06

(52) U.S. Cl. CPC C07D 251/24; C07C 251/24; C07D 471/06; C07C 2603/50

(57) ABSTRACT

The present invention relates to a method of preparing covalent organic frameworks. A gamma ray illuminates monomers for triggering polymerization and producing covalent organic frameworks. The reaction can be performed at normal temperature and pressure. By shortening the reaction time, the method is beneficial to industrial mass production.

### BACKGROUND OF THE INVENTION

The design and application of polymers is an area of considerable attention in modern material science. The principle is to use various connection methods to assemble and connect different small structural units to form one-, two-, or three-dimensional structures. These connection methods include covalent linkage and pi-pi stacking arrangements. Common polymers such as polyethylene, nylon, polyester compounds, or polyamine compounds formed by covalent polymerization using small structural units such as olefins, amines, or organic acids are widely used in modern daily life.

Among the above polymers, the material called “covalent organic framework” has the characteristics of low density, high specific surface area, controllable pore structure, case in adjusting properties, high material strength, stable chemical properties, etc., and can be used in applications such as gas storage, adsorption, photoelectricity, catalysis, or functional devices and other applications. Covalent organic frameworks are considered to be excellent materials after the current metal organic frameworks. Since covalent organic frameworks have the potential to be applied in various fields, many teams have devoted themselves to the research and development of this material.

The research on covalent organic framework materials was originally proposed by Professor Omar M. Yaghi and his team from the University of California, Berkeley. In 2005, the team used phenyl diboronic acid and hexahydroxytriphenylenebenzene to undergo a condensation reaction and a three-dimensional covalent organic framework was synthesized successfully.

So far, preparation methods for the synthesis of covalent organic framework materials have been developed, including solvothermal synthesis, ionothermal synthesis, microwave-assisted synthesis, and mechanochemical synthesis. Among them, the solvothermal synthesis method is the most widely used technology, which is to polymerize the monomer solution for several days in a closed container under high temperature and high pressure environment. However, the disadvantage of this method is that high temperature and high pressure are required, not only the production cost is high, but also the requirements for machinery and equipment are also increased. In addition, batch production in a closed container is difficult to be prepared on a large scale, and the reaction time is too long, which is disadvantageous for large-scale industrial production. It is the goal of current industry research and development to seek a preparation method that have high efficiency under normal reaction conditions.

### SUMMARY OF THE INVENTION

An objective of the present invention is to provide a novel method of preparing covalent organic frameworks. The reaction can be performed at normal temperature and pressure for lowering production costs. By shortening the reaction time, the method is beneficial to industrial mass production.

To achieve the above objective, the present invention provides a method of preparing covalent organic frameworks, which comprises steps of: placing one or more monomer into a container in one or more form; using a gamma ray to illuminate the monomer for a time; and the one or more monomer undergoing a polymerization reaction by the gamma ray and forming a covalent organic framework.

According to an embodiment of the present invention, in the step of placing one or more monomer into a container in one or more form, the one or more monomer is selected from the group consisting of substituted triphenylbenzene, unsubstituted triphenylbenzene, substituted tetraphenylpyrene, and unsubstituted tetraphenylpyrene.

According to an embodiment of the present invention, in the step of placing one or more monomer into a container in one or more form, the one or more form is a solution prepared by using the one or more monomer and one or more solvent or the power form of the one or more monomer.

According to an embodiment of the present invention, the one or more solvent is selected from the group consisting of organic solvent and water.

According to an embodiment of the present invention, the one or more solvent is selected from the group consisting of p-dichlorobenzene (p-DCB), n-butanol (n-BuOH), mesitylene, dioxane, N-methyl pyrrolidone (NMP), isoquinoline, acetic acid, and water.

According to an embodiment of the present invention, in the step of placing one or more monomer into a container in one or more form, the material of the container is plastics, glass, ceramic, or metal.

According to an embodiment of the present invention, in the step of using a gamma ray to illuminate the monomer for a time, the radiation dose rate of the gamma ray is 0.001 to 1000 kGy/h.

According to an embodiment of the present invention, in the step of using a gamma ray to illuminate the monomer for a time, the time is greater than 0 second and smaller than 7 days.

According to an embodiment of the present invention, in the step of the one or more monomer undergoing a polymerization reaction by the gamma ray, the temperature of the polymerization reaction is-200 to 1000° C.

According to an embodiment of the present invention, in the step of the one or more monomer undergoing a polymerization reaction by the gamma ray, the pressure of the polymerization reaction is 0 to 30 atm.

### BRIEF DESCRIPTION OF DRAWINGS

FIG. 1 shows a flowchart of the method of preparing covalent organic frameworks according to the present invention;

FIG. 2A to FIG. 2F show powder X-ray diffraction analysis results on the product of the covalent organic frameworks according to the present invention; and

FIG. 3A to FIG. 3F show experimental results of the covalent organic frameworks according to the present invention.

### DETAILED DESCRIPTION OF THE INVENTION

The preparation methods of covalent organic frameworks according to the prior art require high-pressure and high-temperature production conditions, as well as high-temperature and high-pressure-resistant containers. The production can only be done in small batches. The reaction takes many days. These features make them disadvantageous for industrial mass production.

Different from the preparation methods according to the prior art, the present invention adopts a gamma ray to illuminate the monomers for inducing reactions and forming covalent organic frameworks and hence solving the mass production problem as described above.

In the following description, various embodiments of the present invention are described using figures for describing the present invention in detail. Nonetheless, the concepts of the present invention can be embodied by various forms. Those embodiments are not used to limit the scope and range of the present invention.

According to the present invention, the term “gamma ray” means a type of penetrating electromagnetic radiation produced by the radioactive decay of atomic nuclei. The general frequency is greater than 3×10¹⁹ Hz. In applications, it is expressed by radiation dose rate.

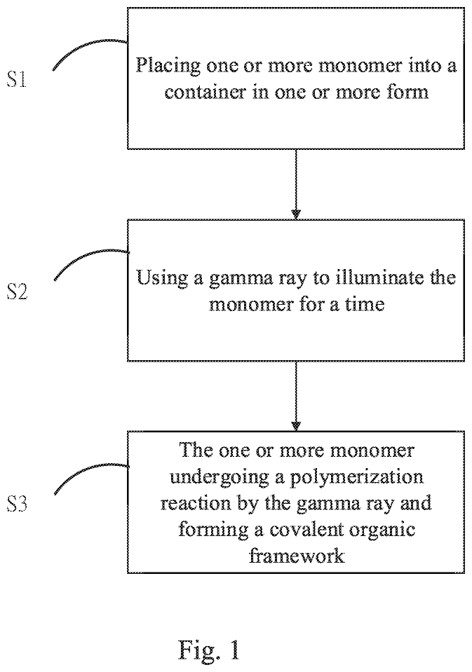

First, please refer to FIG. 1, which shows a flowchart of the method of preparing covalent organic frameworks according to the present invention. The method comprises the following steps:
Step S1: Placing one or more monomer into a container in one or more form;
Step S2: Using a gamma ray to illuminate the monomer for a time; and
Step S3: The one or more monomer undergoing a polymerization reaction by the gamma ray and forming a covalent organic framework.

In the following, the steps will be illustrated in detail.

In the step S1, one or more monomer is placed into a container in one or more form. The one or more monomer used in the preparation method according to the present invention means the small-molecule compound for synthesizing covalent organic frameworks. The one or more monomer is preferably selected from the group consisting of substituted triphenylbenzene, unsubstituted triphenylbenzene, substituted tetraphenylpyrene, and unsubstituted tetraphenylpyrene. More preferably, it is selected from the group consisting of 1,3,5-tris-(4-aminophenyl)triazine (TPT-3NH₂), 2,4,6-Tris(4-formylphenyl)triazine (TPT-3CHO), 1,3,5-tris (4′-aldehyde phenyl) benzene (TPB-3CHO), and 4,4′,4″,4′″-pyrene-1,3,6,8-tetrayl)tetraaniline (Py-4NH₂).

The method of synthesizing 1,3,5-tris-(4-aminophenyl)triazine (TPT-3NH₂) is:

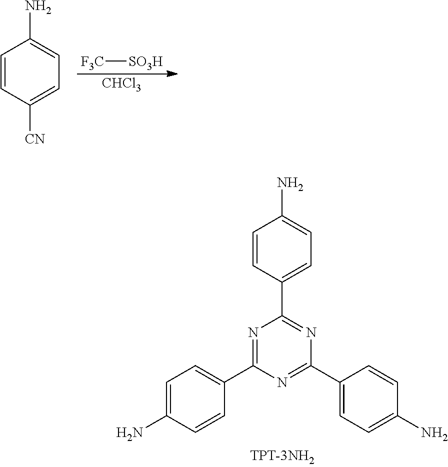

Add 4-aminobenzonitrile (1.5 g, 12.70 mmol) into 20 mL of anhydrous trichloromethane (CHCl₃) to form a suspension and place the suspension into a 100 mL two-neck bottle. After an ice bath in a nitrogen ambient, add 4 mL (0.045 mmol) of trifluoromethanesulfonic acid. The mixed suspension is stirred in the ice bath for 30 minutes and recovered to the room temperature. After stirring at the room temperature for 24 hours, add ice water. Next, ammonia is used to neutralize the suspension. Finally, the vacuum filtering method is used to dry up. Overnight, the white solids TPT-3NH₂ will be produced with a yield of 80%. FT-IR (powder): 3460, 3379, 3321, 3207, 1633, 1606, 1579, 1498, 1433, 1367, 1309, 1180, 1147, 813, 590, 513 cm⁻¹. ¹H NMR (DMSO-d₆, 25° C., 600 MHz): δ=5.89 (s, 6H), 8.36 (d, J=10.2 Hz, 6H), 6.70 (d, J=10.2 Hz, 6H). 13C NMR (DMSO-d₆, 25° C., 500 MHz): 170.45, 153.77, 130.82, 123.56, 113.69.

The method of synthesizing 2,4,6-Tris(4-formylphenyl)triazine (TPT-3CHO) is:

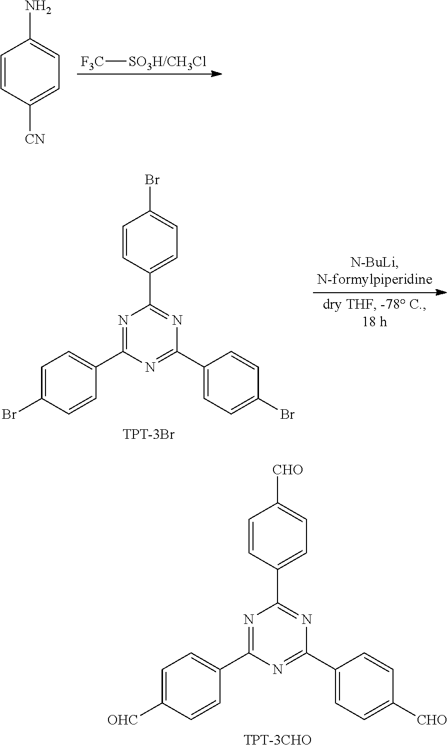

Add 4-aminobenzonitrile (1.5 g, 12.70 mmol) into 20 mL of anhydrous trichloromethane (CHCl₃) to form a suspension and place the suspension into a 100 mL two-neck bottle. After an ice bath in a nitrogen ambient, add 4 mL (0.045 mmol) of trifluoromethanesulfonic acid. The mixed suspension is stirred in the ice bath for 30 minutes and recovered to the room temperature. After stirring at the room temperature for 20 hours, add ice water. Next, ammonia is used to neutralize the suspension. Finally, the vacuum filtering method is used to dry up. Overnight, the white solids TPT-3Br will be produced with a yield of 92%. FT-IR (powder): 2821, 2729, 1706, 1583, 1517, 1419, 1357, 1298, 1201, 1105, 1012, 806, 499 cm⁻¹. ¹H NMR (CDCl₃ and DMSO-d₆, 25° C., 600 MHz): 10.12 (s, 3H), 8.89 (d, J=9.6 Hz, 6H), 8.06 (d, J=9.6 Hz, 6H). 13C NMR (CDCl₃, 25° C., 500 MHz): 192.48, 171.47, 140.77, 139.46, 130.03, 129.80.

Afterwards, add the above product TPT-3Br into 130 mL of anhydrous tetrahydrofuran (THF) to form a suspension and place the suspension into a 250 mL two-neck bottle. Lower the temperature to −78° C. in the nitrogen ambient before adding 7.30 mL (18.6 mmol) of 2.5 M n-butyllithium (n-BuL) hexane solution. The mixed suspension is stirred at −70° C. for 3 hours and recovered to the room temperature. After stirring at the room temperature for 16 hours, add 50 mL of 2.88M hydrochloric acid. Extract the product using chloroform; strip water using anhydrous magnesium sulfate; and filter the product. Finally, use reduced pressure distillation to remove the solvent and purify by centrifugation to give white solids TPT-3CHO with a yield of 80%. FT-IR (powder): 2821, 2729, 1706, 1583, 1517, 1419, 1357, 1298, 1201, 1105, 1012, 806, 499 cm⁻¹. ¹H NMR (CDCl₃ and DMSO-d₆, 25° C., 600 MHz): 10.12 (s, 3H), 8.89 (d, J=9.6 Hz, 6H), 8.06 (d, J=9.6 Hz, 6H). 13C NMR (CDCl₃, 25° C., 500 MHz): 192.48, 171.47, 140.77, 139.46, 130.03, 129.80.

The method of synthesizing 1,3,5-tris (4′-aldehyde phenyl) benzene (TPB-3CHO) is:

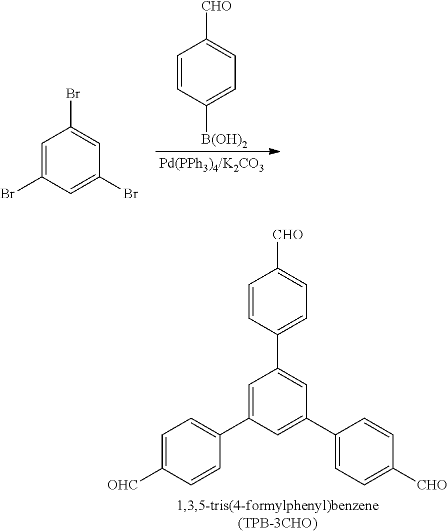 [SMILES: Brc1cc(Br)cc(Br)c1 ; O=Cc1ccc(B(O)O)cc1 ; O=Cc1ccc(-c2cc(-c3ccc(C=O)cc3)cc(-c3ccc(C=O)cc3)c2)cc1 ; "1,3,5-tris(4-formylphenyl)benzene(TPB-3CHO)"]

Place 1,3,5-tribromobenzene (0.75 g, 2.38 mmol), 4-(Dihydroxyboryl)benzaldehyde (2.145 g, 14.32 mmol), tetrakis(triphenylphosphine) palladium (0) (138 mg, 0.12 mmol) and potassium carbonate (1.98 g, 14.32 mmol) into a 100 mL round-bottom flask. After vacuuming for 15 minutes at low pressure, add slowly 30 mL of 1,4-dioxane and 5.5 mL of water in a nitrogen ambient. The mixture is heated at 110° C. for 40 hours and recovered to the room temperature. Add ice water into the mixture to form white precipitates. Vacuum filter the precipitates and rinse using water and methanol for several times. Finally, use the flash column chromatography (SiO₂; EA/n-hexane, 1:9) to purify and giving white solids TPB-3CHO with a yield of 80%.

The method of synthesizing 4,4′,4″,4′″-pyrene-1,3,6,8-tetrayl)tetraaniline (Py-4NH₂) is:

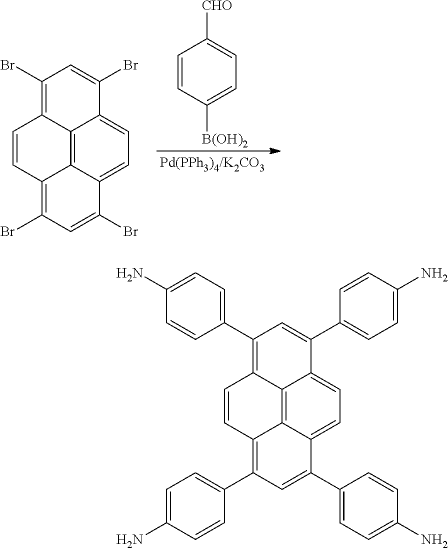

Place 1,3,6,8-tetrabromopyrene (2.0 g, 3.86 mmol), 4-bromoaniline (2.54 g, 18.53 mmol), tetrakis(triphenylphosphine) palladium (0) (452 mg, 0.39 mmol) and potassium carbonate (2.95 g, 21.32 mmol) into a 100 mL round-bottom flask. After vacuuming for 30 minutes at low pressure, add slowly 60 mL of DMF and 10 mL of water in a nitrogen ambient. The mixture is heated at 120° C. for 48 hours. After 1,3,6,8-tetrabromopyrene is exhausted, the temperature is recovered to the room temperature. Add ice water into the mixture to form yellow-green precipitates. Vacuum filter the precipitates and rinse using water and methanol for several times. Finally, purify to give solids Py-4NH₂ with a yield of 80%. 1H NMR (500 MHZ, DMSO-d₆) δ (ppm): 8.12 (s, 4H), 7.79 (s, 2H), 7.35 (d, J=12 Hz, 8H), 6.78 (d, J=12 Hz, 8H), 5.30 (br s, 8H, 4NH₂).

Furthermore, according to the preparation method of the present invention, in the step of placing one or more monomer into a container in one or more form, the one or more form is not limited. Preferably, the one or more monomer is dissolved in one or more solvent to form a solution or the one or more monomer is in the powder form.

In addition, the solvent adopted in the preparation method according to the present invention can dissolve or suspend the monomer as described above. The solvent includes, but not limited to, p-dichlorobenzene (p-DCB), n-butanol (n-BuOH), (Mesitylene), N-methyl pyrrolidone (NMP), isoquinoline, acetic acid, and water.

Besides, since no high-temperature and high-pressure ambient is required and the transparency and thickness has little influence on the transmittance the gamma ray, the material or shape of the container used in the preparation method according to the present invention is not limited. Preferably, plastics, glass, ceramics, or metals can be adopted.

In the step S2, according to the present embodiment, use a gamma ray to illuminate the solution for a time. Since the gamma ray owns extremely high energy, the monomers as described above can be activated to react and forming bonds. According to the present embodiment, the radiation dose rate of the gamma ray is preferably 0.001 to 1000 kGy/h, and more preferably, 0.11 to 0.809 kGy/h. The illumination time decreases as the radiation dose rate increases. Preferably, the illumination time is greater than 0 second and less than 7 days, and more preferably, between 4 minutes and 4 hours.

In the step S3, according to the present embodiment, the one or more monomer undergoes a polymerization reaction by the gamma ray and forming a covalent organic framework. The one or more monomer illuminated by the gamma ray can be activated effectively for undergoing the polymerization reaction. Depending on the type of the adopted monomer, the formed covalent organic framework include several different stereoscopic structures.

According to the present embodiment, the polymerization reaction of using TPB-3NH₂ and TPT-3CHO as monomers to synthesize TPT-TPB-COF is:

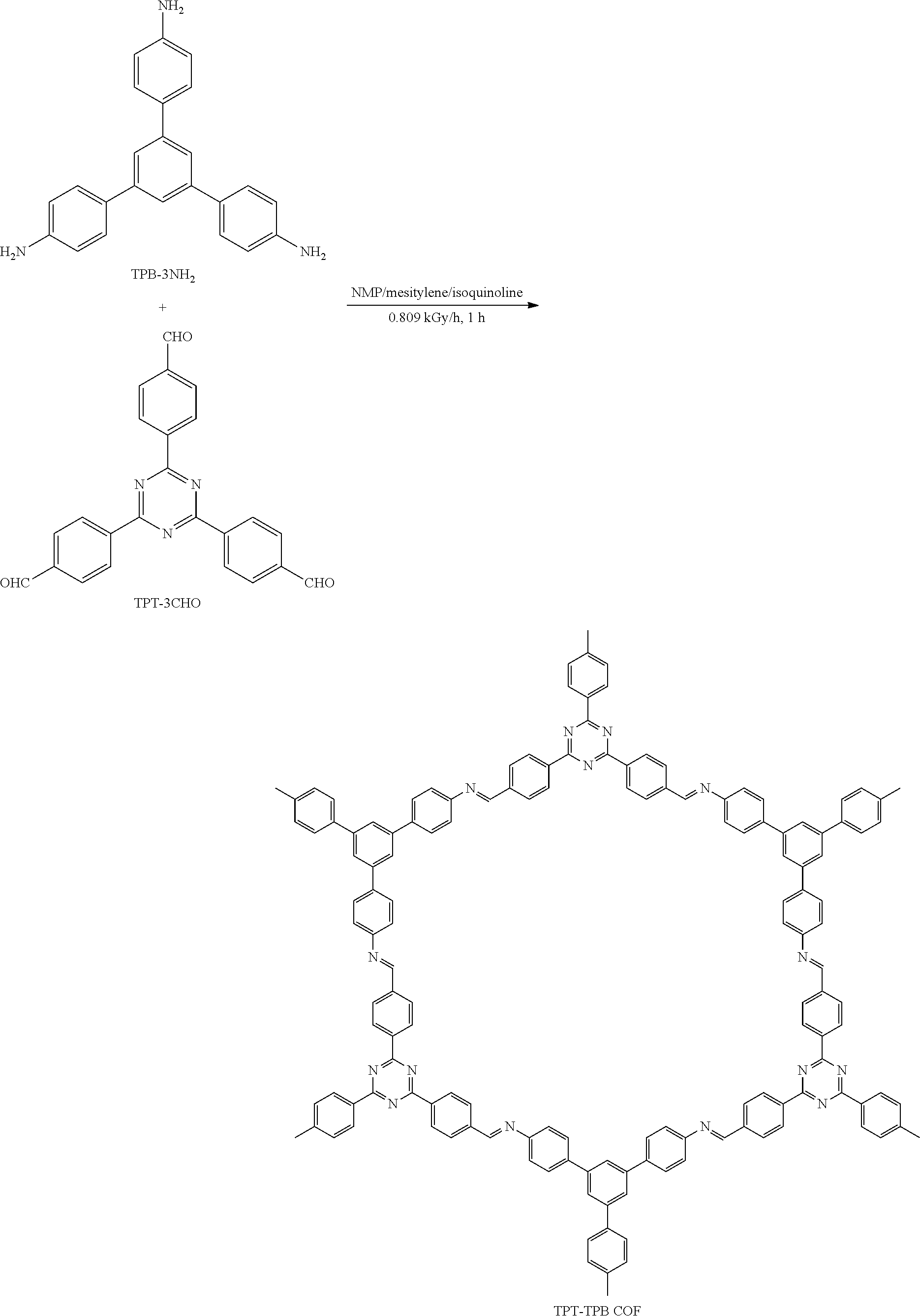

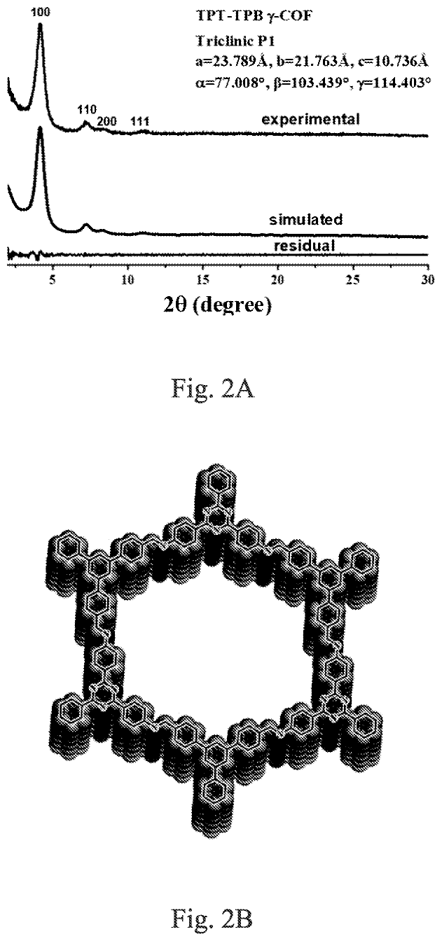

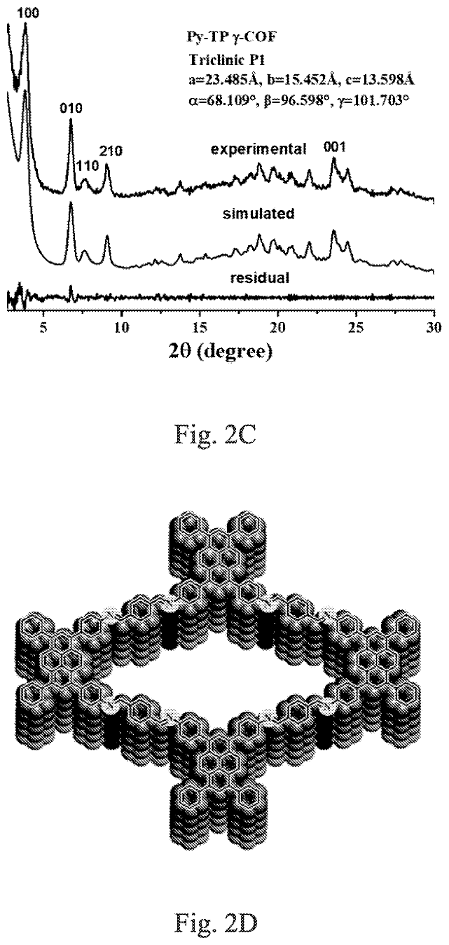

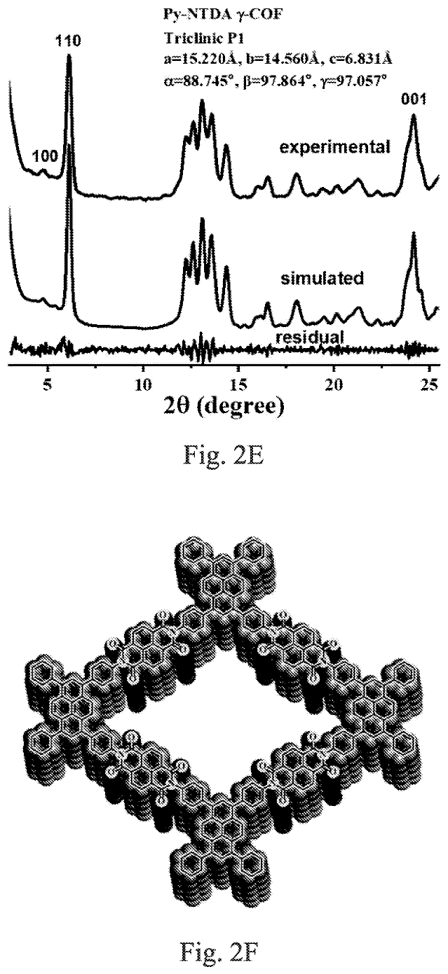

The results of the powder X-ray diffraction (PXRD) analysis of the polymer product are shown in FIGS. 2A and 2B. The polymerization reaction of using TPB-3NH₂ and TPT-3CHO as monomers to synthesize Py-TPT γ-COF is:

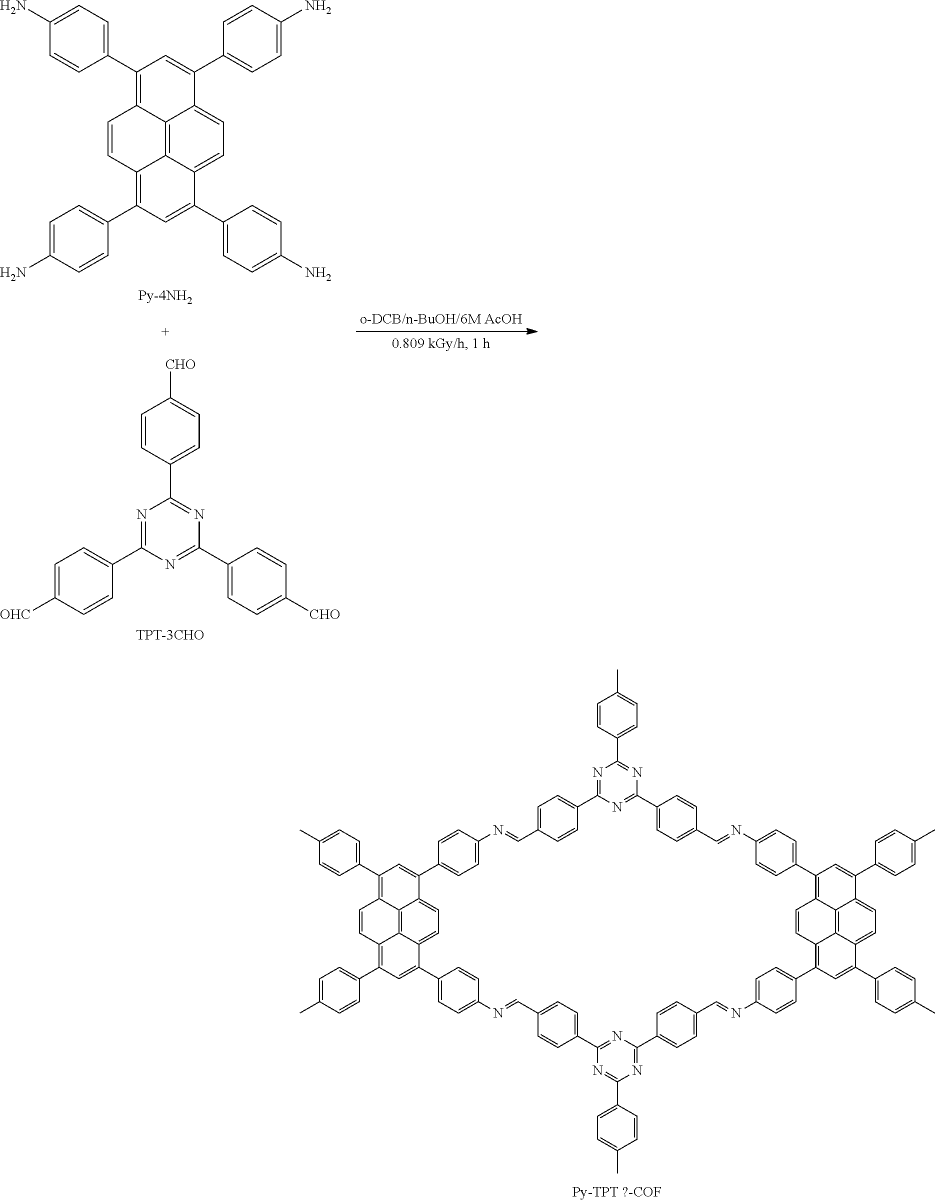

The polymerization reaction of using Py-4NH₂ and TP-2CHO as monomers to synthesize Py-TPT γ-COF is:

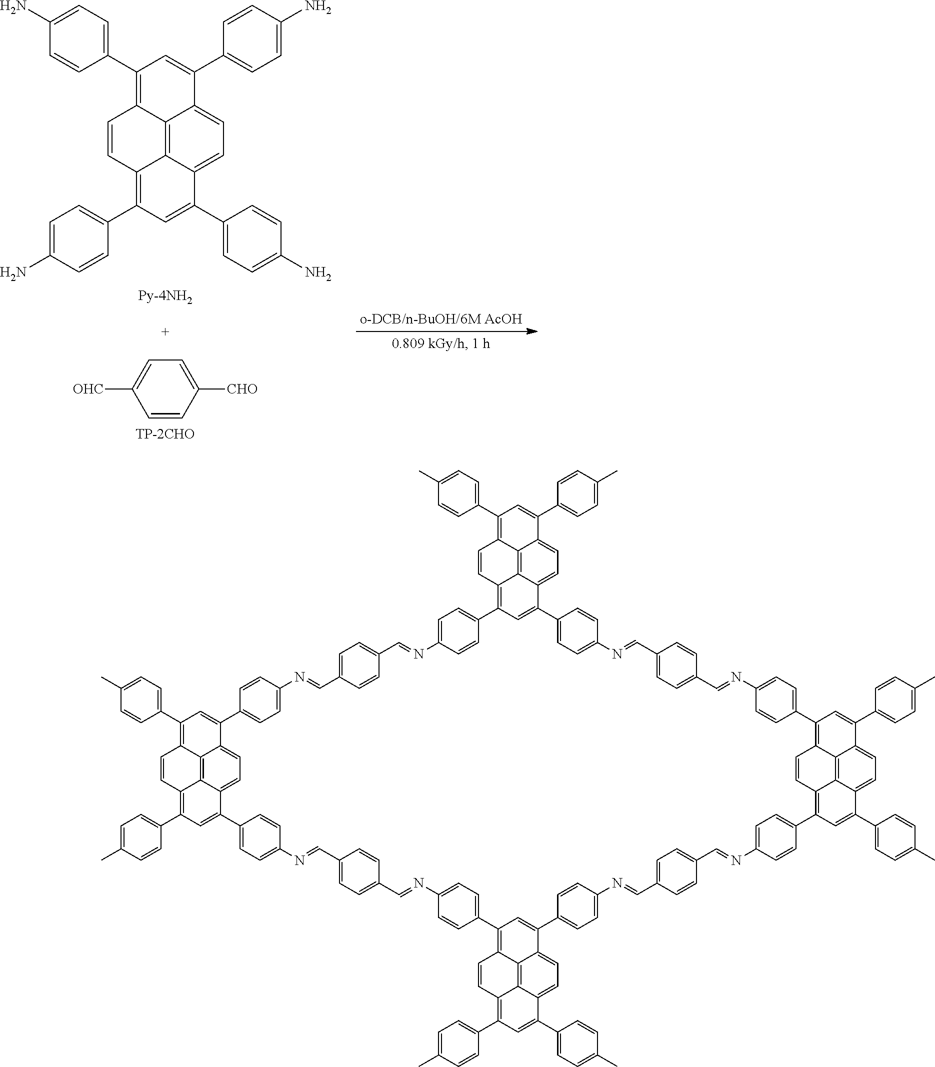

The results of the powder X-ray diffraction (PXRD) analysis of the polymer product are shown in FIGS. 2C and 2D. The polymerization reaction of using Py-4NH₂ and NTDA as monomers to synthesize Py-NTDA-γ-COF is:

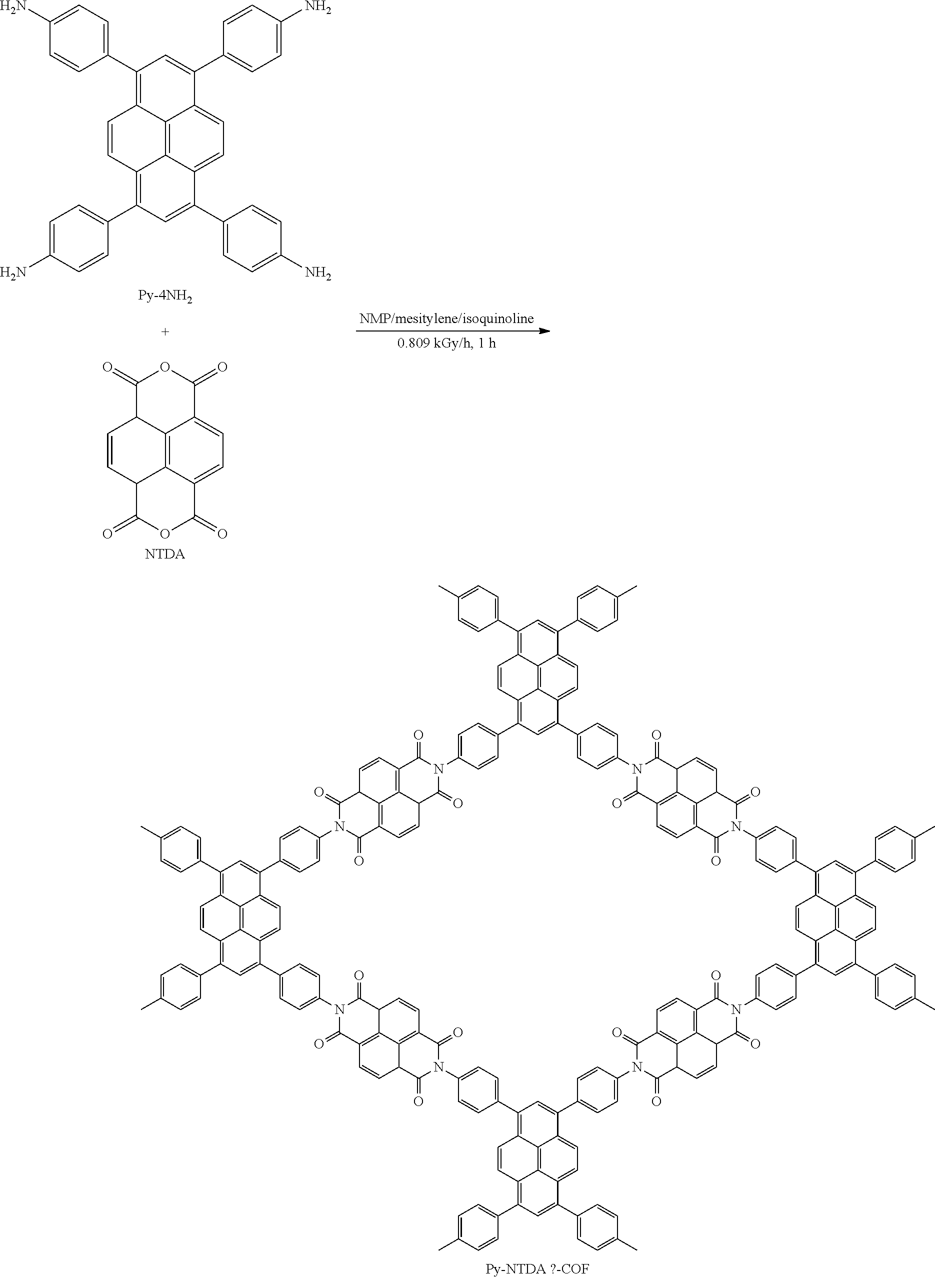

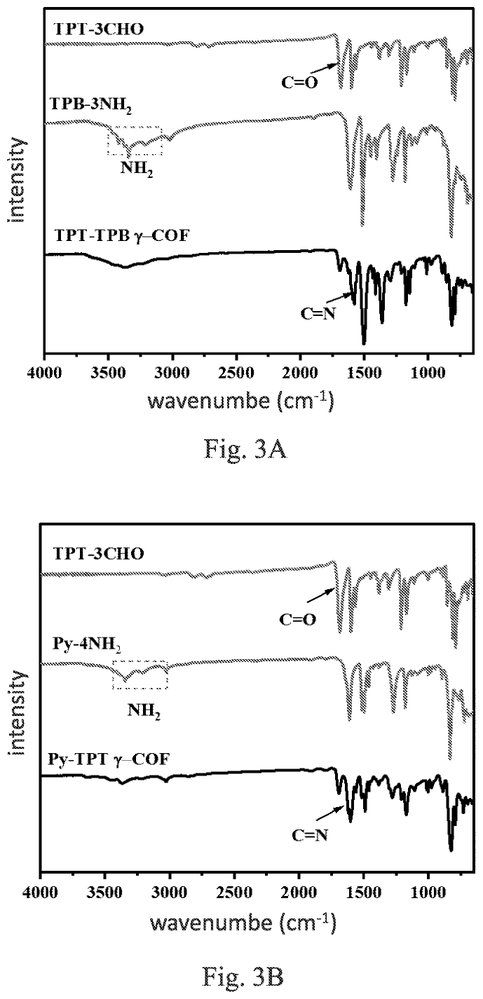

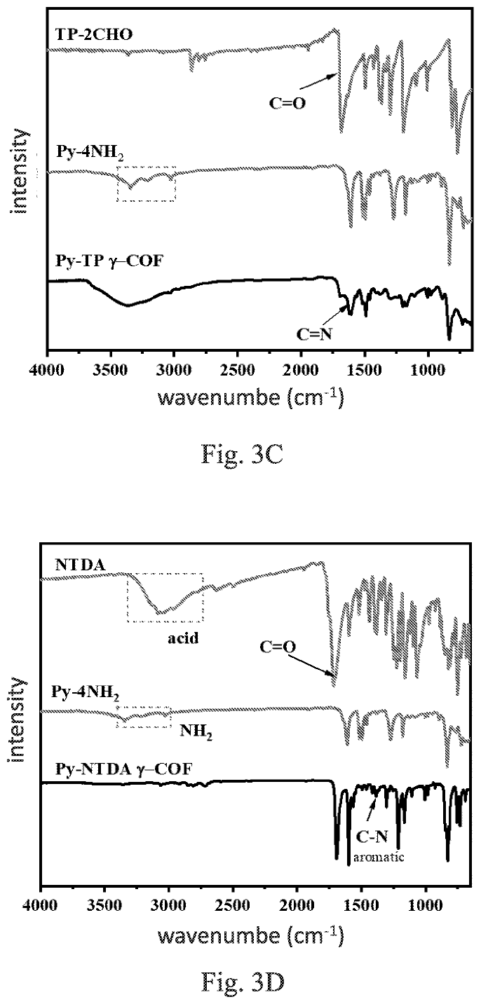

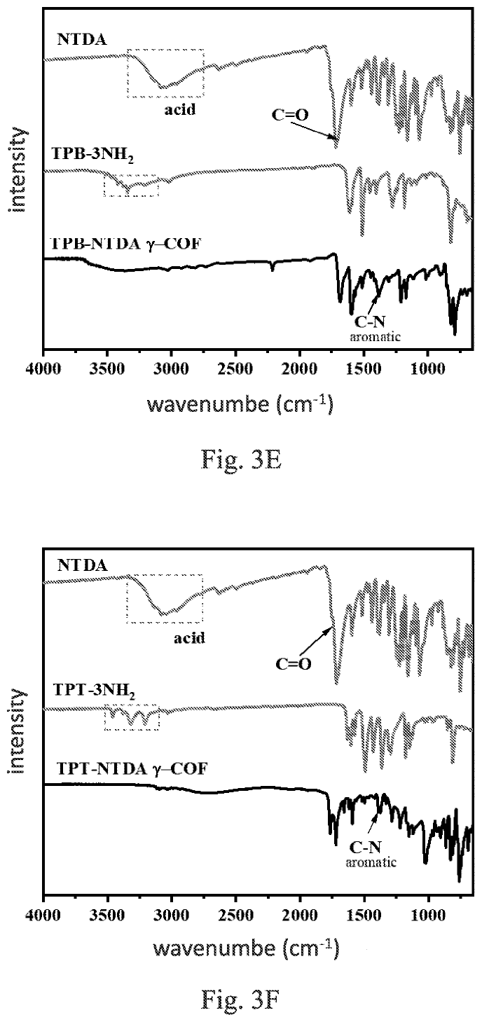

The results of the powder X-ray diffraction (PXRD) analysis of the polymer product are shown in FIGS. 2E and 2F. The synthesized covalent organic framework is examined by the Fourier transform IR spectrum for the structure. The results are shown in FIGS. 3A to 3F. According to the examination results, all the products of the above polymerization reactions exhibit absorption peaks at C═N or C—N bonds, indicating that after illuminating the monomers by the gamma ray, polymerization reactions will occur to produce covalent organic frameworks. According to the above results, the method of preparing covalent organic frameworks according to the present invention indeed can be performed at the normal temperature and pressure. Besides, the reaction time can be shortened, which is beneficial for industrial mass production.

1. A method for preparing covalent organic frameworks, comprising steps of:
placing one or more monomer into a container in one or more form;
using a gamma ray to illuminate said monomer for a time; and
said one or more monomer undergoing a polymerization reaction by said gamma ray and forming a covalent organic framework.

2. The method for preparing covalent organic frameworks of claim 1, wherein said step of placing one or more monomer into a container in one or more form, said one or more monomer is selected from the group consisting of substituted triphenylbenzene, unsubstituted triphenylbenzene, substituted tetraphenylpyrene, and unsubstituted tetraphenylpyrene.

3. The method for preparing covalent organic frameworks of claim 1, wherein said step of placing one or more monomer into a container in one or more form, said one or more form is a solution form prepared by using said one or more monomer and one or more solvent, or the powder form of said one or more monomer.

4. The method for preparing covalent organic frameworks of claim 3, wherein said one or more solvent is selected from the group consisting of organic solvent and water.

5. The method for preparing covalent organic frameworks of claim 4, wherein said one or more solvent is selected from the group consisting of p-dichlorobenzene (p-DCB), n-butanol (n-BuOH), mesitylene, dioxane, N-methyl pyrrolidone (NMP), isoquinoline, acetic acid, and water.

6. The method for preparing covalent organic frameworks of claim 1, wherein said step of placing one or more monomer into a container in one or more form, the material of said container is plastics, glass, ceramic, or metal.

7. The method for preparing covalent organic frameworks of claim 1, wherein said step of using a gamma ray to illuminate said monomer for a time, the radiation dose rate of said gamma ray is 0.001 to 1000 kGy/h.

8. The method for preparing covalent organic frameworks of claim 1, wherein said step of using a gamma ray to illuminate said monomer for a time, said time is greater than 0 second and smaller than 7 days.

9. The method for preparing covalent organic frameworks of claim 1, wherein said step of said one or more monomer undergoing a polymerization reaction by said gamma ray, the temperature of said polymerization reaction is −200 to 1000° C.

10. The method for preparing covalent organic frameworks of claim 1, wherein said step of said one or more monomer undergoing a polymerization reaction by said gamma ray, the pressure of said polymerization reaction is 0 to 30 atm.
